(19) United States
(12) Patent Application Publication
(10) Pub. No.: US 2017/0188822 A1
(43) Pub. Date: Jul. 6, 2017

(54) System And Method For Real-Time Eye Tracking For A Scanning Laser Ophthalmoscope

(71) Applicant: University of Rochester, Rochester, NY (US)

(72) Inventor: Qiang Yang, Rochester, NY (US)

(21) Appl. No.: 15/313,727

(22) PCT Filed: Jul. 14, 2015
(86) PCT No.: PCT/US15/40399

§ 371 Date: Nov. 23, 2016

Related U.S. Application Data
(60) Provisional application No. 62/024,144, filed on Jul. 14, 2014.

(51) Int. Cl. A61B 3/10; G06F 3/01; G06T 7/73; A61B 3/00; G06T 7/246

(52) U.S. Cl. CPC A61B 3/1025; A61B 3/102; A61B 3/0025; G06T 7/248; G06T 7/73; G06F 3/013; G06T 2207/30201

(57) ABSTRACT

Systems and methods for real-time eye tracking using a SLO or other imaging device are described. The systems and methods provide robust and accurate image-based eye tracking for both small and large field SLO, with or without adaptive optics. Methods for rapidly re-locking the tracking of a subject's eye position after a microsaccade, a blink, or some other type of interference with image tracking are also described.

### CROSS-REFERENCE TO RELATED APPLICATIONS

This application claims priority to PCT international application No. PCT/US15/40399 filed on Jul. 14, 2015, which claims priority to U.S. provisional application No. 62/024,144 filed on Jul. 14, 2014, both of which are incorporated herein by reference in their entireties.

### STATEMENT REGARDING FEDERALLY SPONSORED RESEARCH OR DEVELOPMENT

This invention was made with government support under Grant Nos. EY001319 and EY014375 awarded by National Institutes of Health. The government has certain rights in the invention.

### BACKGROUND

Scanning laser ophthalmoscopy uses horizontal and vertical mirrors to scan and image a region of a subject's retina. Adaptive optics can be used to remove optical aberrations from images obtained using a scanning laser ophthalmoscope (SLO). However, fixational eye movement can cause the small field of view (FOV) of an adaptive optics scanning light ophthalmoscope (AOSLO) to shift as the eye moves. Offline registration is generally used to average multiple image frames to obtain a high resolution image with a high signal-to-noise ratio. In patients with poor fixation, large eye motion can cause offline registration to fail when the images to be registered, i.e., target images, move out of the reference image.

Live retinal images from a scanning light ophthalmoscope contain a high percentage of low-contrast and dark regions, even if the optical system has been optimized. This problem can be due to a variety of reasons. For example, the pupil size of the subject can change due to fatigue, variation of the axial position of the subject can cause defocused retinal images, and some patients have low structural information on their retinas due to eye disease. In a real-time image-based eye tracking system, a tracking algorithm is used to control one or multiple tracking mirrors based on the tracking signals retrieved from low-contrast images. These low-contrast images can introduce artifacts or noises on the tracking signals because the tracking algorithm does not always return high-fidelity eye motion signals from different images. When these artifacts are applied to the tracking signals, they can make the tracking mirror jitter, resulting in tracking failure. Ideally, the motion of the tracking mirror should be suspended, i.e., the position of the tracking mirror should be maintained at the existing position, once an artifact is identified. However, in practical implementation, where eye motion can include blinks and saccades, it is difficult to identify and distinguish true eye motion from an artifact.

Further, there is currently no effective system or method for performing high-resolution eye tracking and registration in real-time. Real-time eye tracking has been attempted by using a wide FOV line-scanning system. However, real-time tracking using such a wide FOV hardware-based system does not work consistently. Further, in such systems there is no communication between the wide FOV system and the small FOV system, and the small FOV system is not used for additional real-time tracking to remove residual image motion. Accordingly, such systems do not perform real-time small FOV, i.e., high resolution, eye tracking and registration.

Thus, there is a need in the art for a system and method of high resolution eye-tracking for use with a scanning light ophthalmoscope, particularly one that can distinguish between artifacts in low-contrast tracking images and actual eye motion in the subject with high accuracy.

### SUMMARY

Described herein are systems and methods for real-time eye tracking using scanning laser ophthalmoscopy. In one embodiment, the system is a scanning laser ophthalmoscopy system, comprising: a wide field of view scanning laser ophthalmoscope (SLO) having a controller, a beam splitter, a first tracking mirror, a second tracking mirror, and a small field of view imaging apparatus having a controller, wherein the beam splitter is configured to split a beam of light backscattered from a subject's eye into a first beam and a second beam, such that the first beam is incident on the wide field of view SLO, and the second beam is incident on the first tracking mirror; the second beam is further reflected by the first tracking mirror onto the small field of view apparatus via the second tracking mirror; the wide field of view SLO controller is communicatively coupled with the first tracking mirror; the small field of view apparatus controller is communicatively coupled with the second tracking mirror; and the system compensates for a motion of the subject's eye during eye tracking by moving the first tracking mirror via the wide field of view SLO controller, moving the second tracking mirror via the small field of view apparatus controller, or both.

In another embodiment, the system comprises: a wide field of view scanning laser ophthalmoscope (SLO), a beam splitter, a small field of view imaging apparatus, a controller communicatively coupled with the wide field of view SLO and the small field of view imaging apparatus, and a tracking mirror communicatively coupled with the controller, wherein the tracking mirror is configured to receive a beam of light backscattered from a subject's eye; the beam of light received by the tracking mirror is reflected onto the beam splitter; the beam splitter splits the beam of light into a first beam and a second beam, such that the first beam is incident on the wide field of view SLO, and the second beam is incident on the small field of view imaging apparatus; and the system compensates for a motion of the subject's eye during eye tracking by moving the tracking mirror via the controller.

In yet another embodiment, the system comprises: a wide field of view scanning laser ophthalmoscope (SLO), a beam splitter, a small field of view imaging apparatus, a controller communicatively coupled with the wide field of view SLO and the small field of view imaging apparatus, and a tracking mirror communicatively coupled with the controller, wherein the beam splitter is configured to receive a beam of light backscattered from a subject's eye; the beam splitter splits the beam of light into a first beam and a second beam, such that the first beam is incident on the wide field of view SLO, and the second beam is incident on the tracking mirror; and the system compensates for a motion of the subject's eye during eye tracking by moving the tracking mirror via the controller.

In one embodiment, the small field of view apparatus is a small field of view SLO. In one embodiment, the small field of view apparatus is an adaptive optics scanning light ophthalmoscope (AOSLO). In one embodiment, the small field of view apparatus is an optical coherence tomography (OCT) apparatus. In one embodiment, the field of view of the wide field of view SLO is in the range of about 10 to 30 degrees. In one embodiment, the field of view of the small field of view apparatus is in the range of about 1 to 2 degrees. In one embodiment, moving the first tracking mirror via the wide field of view SLO controller can compensate for an eye motion of about ±3°. In one embodiment, moving the second tracking mirror via the small field of view apparatus controller can compensate for an eye motion of about ±3°. In one embodiment, moving the single tracking mirror via the controller can compensate for an eye motion of about ±6°.

In one embodiment, the method is a method of real-time eye tracking using a small field of view imaging system, comprising: obtaining a reference image of at least a portion of a subject's retina, dividing at least a portion of the reference image into one or more strips, sending the one or more reference strips to a microprocessor, obtaining a high resolution target image of at least a portion of the subject's retina, dividing at least a portion of the target image into one or more strips, sending the one or more target strips to a host microprocessor, sending the one or more target strips from the host microprocessor to a graphics microprocessor, wherein each target strip is correlated with a reference strip, returning at least one output parameter from the graphics microprocessor to the host microprocessor, wherein the at least one output parameter corresponds to the motion of the target strip compared to the reference strip, and registering the target image to the reference image based on the at least one output parameter.

In one embodiment, the at least one output parameter is a correlation coefficient. In one embodiment, the at least one output parameter is an x translation or a y translation. In one embodiment, the time for correlating each target strip with a reference strip is less than about 0.2 milliseconds. In one embodiment, the total latency time from obtaining the reference image to registering the target frame to the reference image is less than about 2 milliseconds. In one embodiment, the reference image is obtained from a wide field of view SLO. In one embodiment, the target image is obtained from a small field of view imaging apparatus. In one embodiment, the small field of view imaging apparatus is a small field of view SLO. In one embodiment, the small field of view imaging apparatus is an AOSLO. In one embodiment, the small field of view imaging apparatus is an OCT apparatus. In one embodiment, the target image is not registered to the reference image if the target image corresponds to a saccade or blink of the subject's eye.

In one embodiment, the direction of the wide field of view SLO fast-scanning axis is perpendicular to the small field of view apparatus fast-scanning axis, and the wide field of view SLO slow-scanning axis is perpendicular to the small field of view apparatus slow-scanning axis.

### BRIEF DESCRIPTION OF THE DRAWINGS

The following detailed description of embodiments will be better understood when read in conjunction with the appended drawings. It should be understood, however, that the embodiments are not limited to the precise arrangements and instrumentalities shown in the drawings.

FIG. 1 is a set of images showing an example of image registration failure in images from an AOSLO.

FIG. 2 is a set of SLO images illustrating the field of motion differences between an AOSLO image with and without the real-time tracking.

FIG. 3 is a schematic diagram of an exemplary embodiment of an eye tracking system.

FIG. 4 is a schematic diagram of another exemplary embodiment of an eye tracking system.

FIG. 5 is a schematic diagram of yet another exemplary embodiment of an eye tracking system.

FIG. 6 is a graph showing data for fixational eye motion in a patient with the disease of cone-rod dystrophy.

FIG. 7 is a graph of image motion data corresponding to images of an eye of a patient with cone-rod dystrophy that was tracked with an embodiment of the system.

FIG. 8 is a single frame of a retinal image from a wide FOV SLO from the eye of a patient with cone-rod dystrophy.

FIG. 9 is a single frame of a retinal image from a wide FOV SLO from the eye of a patient with cone-rod dystrophy showing the size of an exemplary target image (marked with h) and the reference image (marked with II).

FIG. 10 is a set of retinal images from the eye of a patient with cone-rod dystrophy. FIG. 10A is a reference image, featuring strips used for cross-correlation during eye tracking. FIG. 10B is a target image, also featuring strips used for cross-correlation during eye tracking.

FIG. 11 is a schematic diagram of an exemplary embodiment of the electronics system for a wide FOV system.

FIG. 12 is a drawing representing the wide (circle) and small (any square defined by 4 small squares) field of view for an exemplary process of real-time retinal montaging.

FIG. 13, comprising FIGS. 13A and 13B, is a flow chart representing an exemplary eye tracking algorithm.

### DETAILED DESCRIPTION

It is to be understood that the figures and descriptions have been simplified to illustrate elements that are relevant for clear understanding, while eliminating, for the purpose of clarity, many other elements found in the field of image-based eye tracking and scanning-based imaging systems. Those of ordinary skill in the art may recognize that other elements and/or steps are desirable and/or required in implementing systems and methods described herein. However, because such elements and steps are well known in the art, and because they do not facilitate a better understanding, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art. Any methods and materials similar or equivalent to those described herein can be used in the practice for testing of the systems and methods described herein. In describing and claiming the systems and methods, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. Preferably, the patient, subject or individual is a mammal, and more preferably, a human.

Ranges: throughout this disclosure, various aspects can be presented in a range format. It should be understood that the description in range format is merely for convenience and brevity and should not be construed as an inflexible limitation on the scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Described herein are systems and methods for real-time eye tracking using a SLO. The systems and methods provide robust and accurate image-based eye tracking for both small and large field SLO, with or without adaptive optics. The systems and methods are particularly useful for performing eye tracking and registration of high resolution images, i.e., tracking of images from an AOSLO or other small FOV system. Methods for rapidly re-locking the tracking of a subject's eye position after a microsaccade, a blink, or some other type of interference with image tracking are also described. In certain embodiments, the systems and methods disclosed herein can include the use of laser systems and delivery methods, such as those disclosed in U.S. provisional application No. 62/024,140 filed on Jul. 14, 2014, titled “Real-Time Laser Modulation and Delivery in Ophthalmic Devices for Scanning, Imaging, and Laser Treatment of the Eye”, incorporated herein by reference.

Eye tracking requires image registration, which involves relating and aligning the features in a target image with the corresponding features in a reference image. Image registration can be performed “off-line,” wherein a series of high resolution target images are made and then later registered to the reference image. Image registration can also be performed in real-time, wherein features on target images are continuously mapped or registered to the reference image as each target image is being produced. Accurate real-time image registration in ophthalmoscopy is significantly more difficult than off-line registration for a number of reasons. For example, eye motion in the subject can interfere with or prevent accurate image tracking. Further, the light-absorbing nature of a subject's retina generally results in images of the retina having low resolution features. The low resolution of these features make them difficult to track and can result in artifacts being confused with features of the subject's eye.

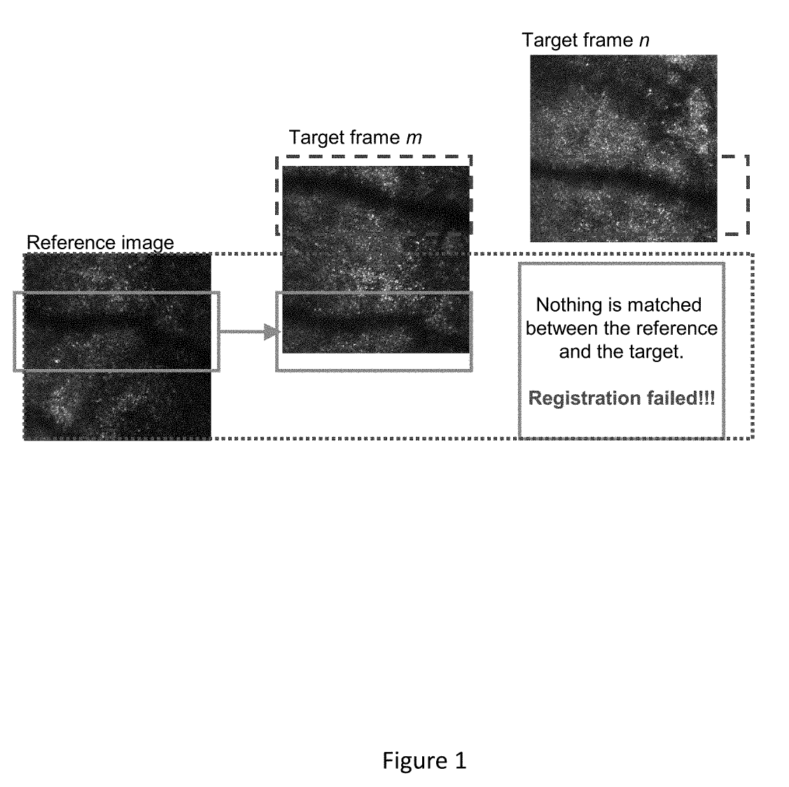

Two types of systems can be used for eye tracking in ophthalmoscopy: a wide FOV system such as a SLO, operating within about 10 to 30 degrees, or a small FOV system such as an AOSLO, operating within about 1 to 2 degrees. A wide FOV SLO is capable of covering large eye motion, but it generally does not have high spatial resolution. An AOSLO has high spatial resolution, but frequently suffers from “frame out,” where the target frame moves out of the reference frame and causes image registration to fail. For example, FIG. 1 illustrates the difficulty in maintaining image registration when using a small FOV system. FIG. 1 is a set of example images from an AOSLO system with a 1.5°×1.5° FOV. Offline image registration was successful for target frame m, but failed for target frame n because it moved out of the mapping area of the reference frame. In this example, the movement of the target image out of the mapping area of the reference frame was caused by constant eye motion in the subject. Accordingly, it is difficult to effectively implement either wide or small FOV SLO alone in real-time in high resolution because a wide FOV SLO has insufficient spatial resolution and a small FOV SLO suffers from consistent failure of image registration. Therefore, image registration with these systems is typically performed off-line by sampling and processing large volumes of videos, which can be inefficient and time-consuming, e.g., on the order of several hours or more.

The systems and methods dramatically reduce the processing time required for image registration, thus enabling the ability to perform image registration in real-time. In one embodiment, the system combines a wide FOV SLO and an AOSLO into a hybrid tracking system that includes at least one tracking mirror for removing large eye motion on the AOSLO. In this embodiment, a signal corresponding to large eye motion is obtained from the wide FOV system, which has low resolution. After correction is applied via the one or more tracking mirrors, the residual eye motion on the small FOV system (AOSLO) is reduced to about 20-50 micrometers, which can be efficiently and quickly registered using a fast GPU registration algorithm.

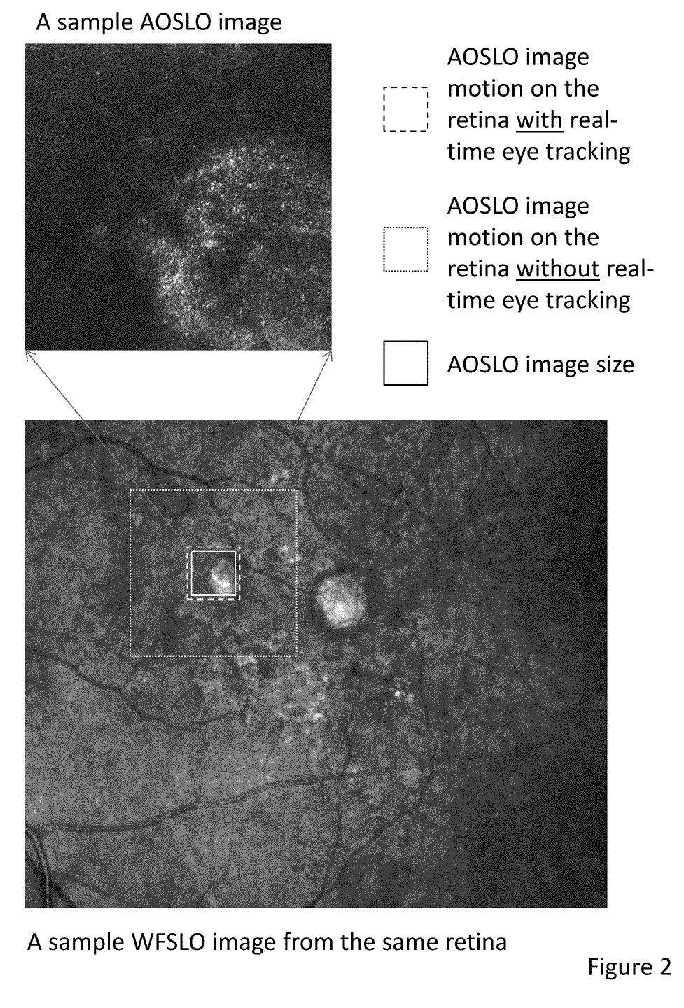

In a diseased eye with poor fixation, the eye typically moves about 1 mm to 3 mm. A 1.5°×1.5° image size from an AOSLO is equivalent to about 0.4 mm-0.6 mm, depending on the axial length of individual eyes and also potentially on other system parameters. This means that motion of an AOSLO image will randomly move about 2-6 times the field size without real-time optical eye tracking, as shown in the dotted rectangle in FIG. 2. With the assistance of optical eye tracking, the residual image motion is reduced to the dashed rectangle in FIG. 2 which is about 20-50 μm.

The advantages of the system include: integration of the small FOV system and the wide FOV system; high resolution images from the small FOV system with residual eye motion can be registered and montaged in real time; and root mean square (RMS) error in the image registration can be reduced to less than about 200-400 nanometers. Accordingly, retinal positions can be tracked efficiently and accurately inside the wide FOV, and the need for the time-consuming post-processing of large volumes of videos is eliminated.

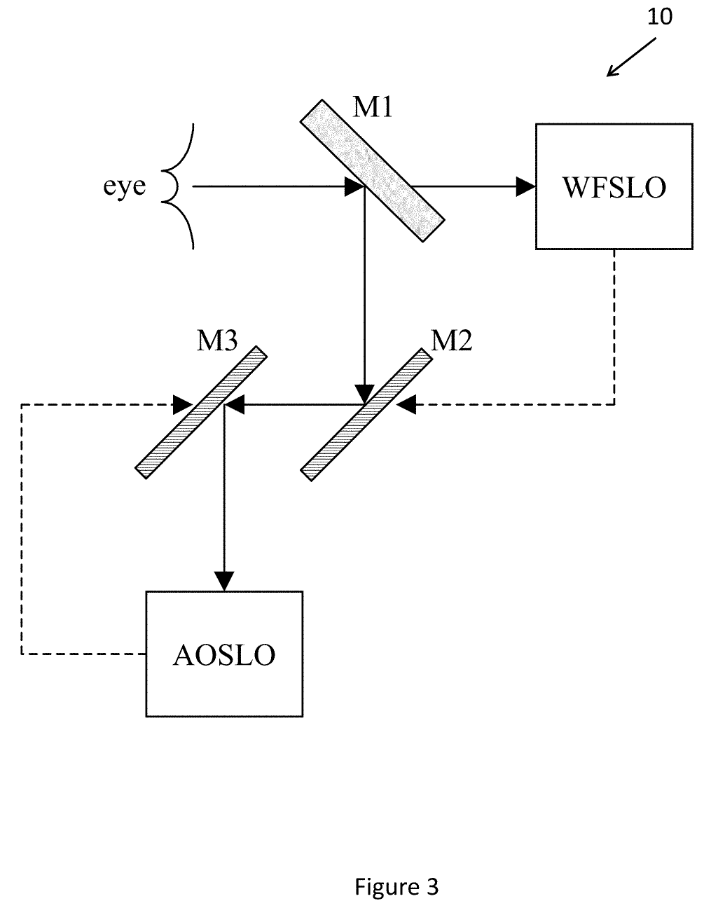

One embodiment of the system is shown in FIG. 3. This system is based on a multi-scale method that can be used to optically stabilize the imaging FOV of a small FOV SLO, for example to compensate for image motion caused by eye motion in the subject being examined. In one embodiment, the small FOV SLO is an AOSLO. In another embodiment, the small FOV SLO does not include adaptive optics. In yet another embodiment, a type of high resolution imaging system other than a SLO can be controlled, for example an optical coherence tomography (OCT) system. The optical system 10 includes a beam splitter M1, a first tracking mirror M2, and a second tracking mirror M3. System 10 also includes a wide FOV FSLO (WFSLO) and an AOSLO. In this embodiment, first tracking mirror M2 is controlled by the WFSLO, and the second tracking mirror M3 is controlled by the AOSLO. Accordingly, tracking mirror M2 is used for course-scaled tuning to compensate for large eye motion via a motion signal sent from the WFSLO, while the second tracking mirror M3 is used for fine-tuned image motion via a motion signal sent from the AOSLO. In this embodiment, both M2 and M3 are able to separately compensate for an eye motion of ±3°. Therefore, M2 and M3 in combination can compensate up to ±6° eye motion, which is sufficient for most fixational eye movements, even in eyes with poor fixation.

Eye motion can be defined as R(t), which is a function of time t. In the system shown in FIG. 3, the WFSLO will detect any eye motion R(t) of the subject's eye within the wide FOV. A tracking algorithm is used to determine the amount, if any, of motion that must be applied to mirror M2 to compensate for the detected eye motion R(t). The WFSLO then sends a signal to the tracking mirror M2 to cause a compensation motion in M2 based on R(t). The motion of M2 can be defined as A(t). Therefore, the residual image motion appearing on M3 will be,

R(t)−A(t) (1)

In the loop of M1-WFSLO-M2, the tracking mirror M2 is working in an open loop because the WFSLO controls the motion of M2, but does not detect the effects of any motion of M2. At the same time, tracking mirror M3 works in a closed loop with the AOSLO because the AOSLO detects residual image motion by dynamically adjusting M3 to compensate for the residual motion R(t)−A(t) after the correction of M2. If the motion of M3 is defined as B(t), the residual image motion on the AOSLO will be the amount of,

R(t)−A(t)−B(t) (2)

which is detected by an AOSLO tracking algorithm.

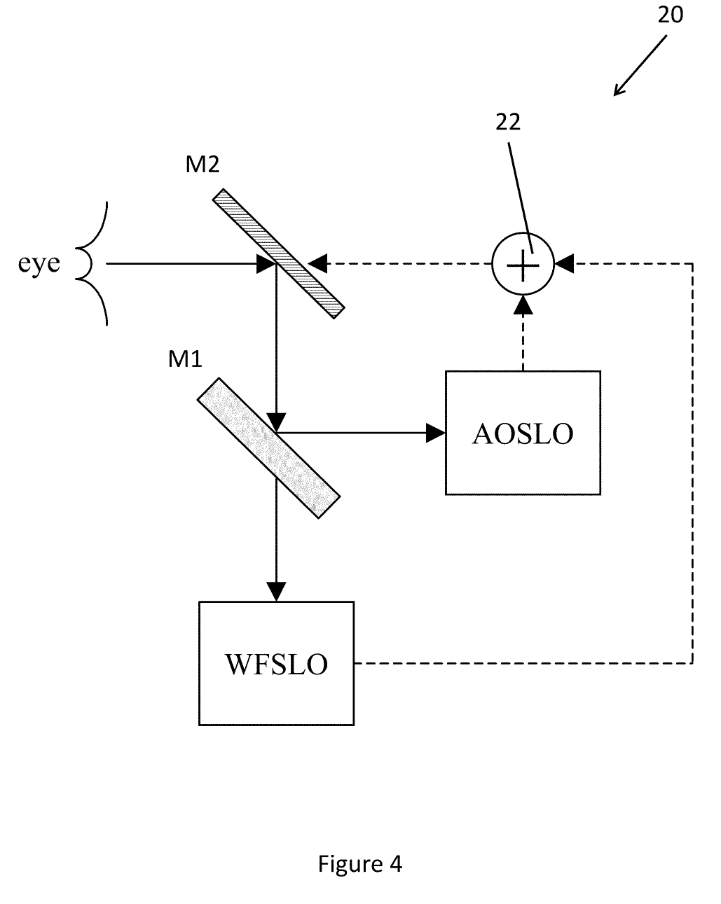

Another embodiment of an eye tracking control system is shown in FIG. 4. System 20 is a simplified tracking control system, where a single tracking mirror M2 is employed to receive control signals from both the small FOV, i.e., the AOSLO, and the WFSLO via a controller 22. System 20 also includes a beam splitter M1. Although the configuration of system 20 eliminates the need for a second tracking or steering mirror, system 20 requires significantly higher quality optical components in comparison to system 10 in order to maintain a similar quality of image tracking.

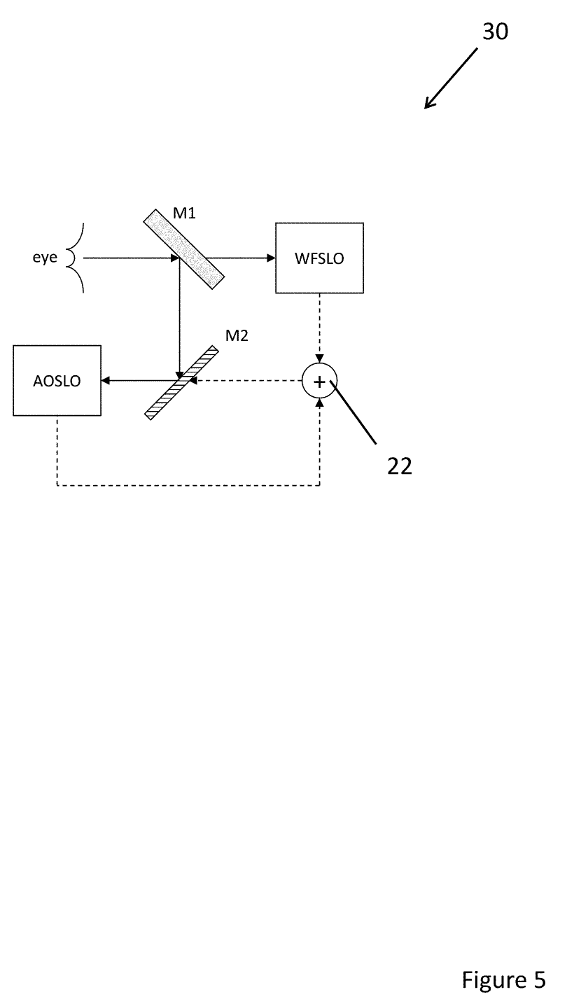

Yet another embodiment of an eye tracking system is shown in FIG. 5. System 30 is also a simplified tracking control system, where a single tracking mirror M2 is employed to receive control signals from both the small FOV, i.e., the AOSLO, and the WFSLO via a controller 22. System 30 also includes a beam splitter M1. In this embodiment, backscattered light from the eye first passes through beam splitter M1 rather than being incident first on tracking mirror M2, as in system 20. In this regard, the combination of AOSLO closed-loop tracking and WFSLO open-loop tracking can be implemented with a single tracking mirror M2, provided that M2 has sufficient dynamic range to compensate for eye motion.

The embodiments shown in FIGS. 3 through 5 can include other components necessary for operation. It is contemplated herein that a person skilled in the art would readily understand, and be able to identify, such components as standard components known in the art. For example, the tracking mirrors described herein would require a mechanism for moving the mirrors based on a control signal. Such a mechanism could include a component for receiving a control signal, and a motor or driver for moving the mirror.

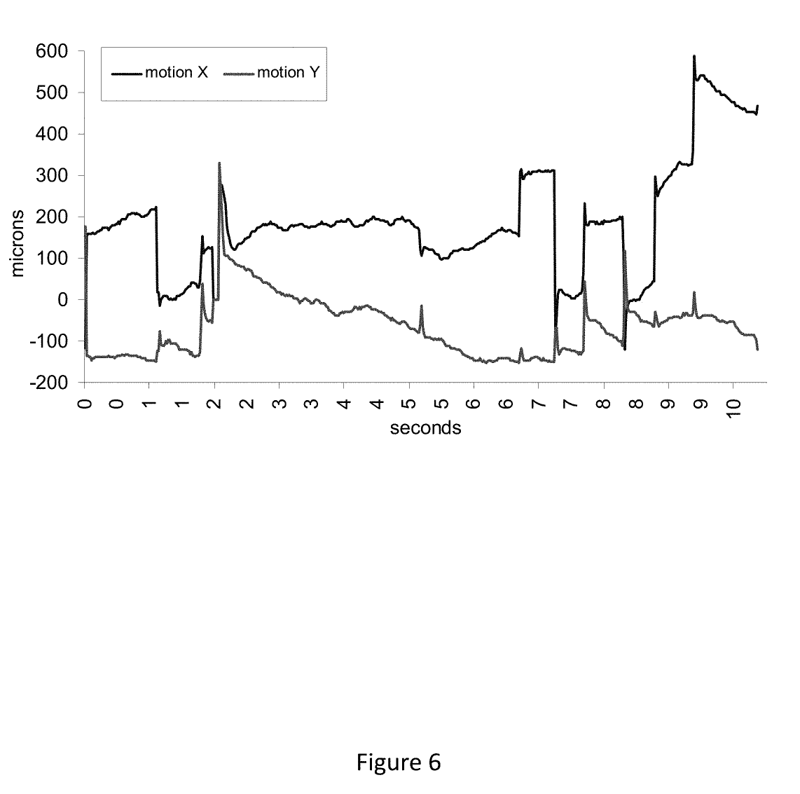

In another aspect, the systems and methods can distinguish true eye motion signals from artifacts present in the target images. Referring to FIG. 6, a graph representing typical fixational eye motion in a patient having the disease of cone-rod dystrophy is shown. The curves in FIG. 6 have a number of spikes. Some of these spikes correspond to true eye motion signals caused by microsaccades. However, some of the spikes correspond to motion artifacts caused by the low contrast of the captured images, i.e., the spikes correspond to false eye motion signals. When these motion artifacts are treated by the system as actual tracking signals, the tracking mirror is moved unnecessarily, i.e., it jitters, which results in tracking failure. In the systems and methods, the tracking mirror is generally not moved in relation to these motion artifacts, i.e., the position of the mirror is held constant when the system identifies a motion as a false eye motion signal. Therefore, a key element of the systems and methods is a robust tracking algorithm that is used to distinguish true eye motions from artifacts.

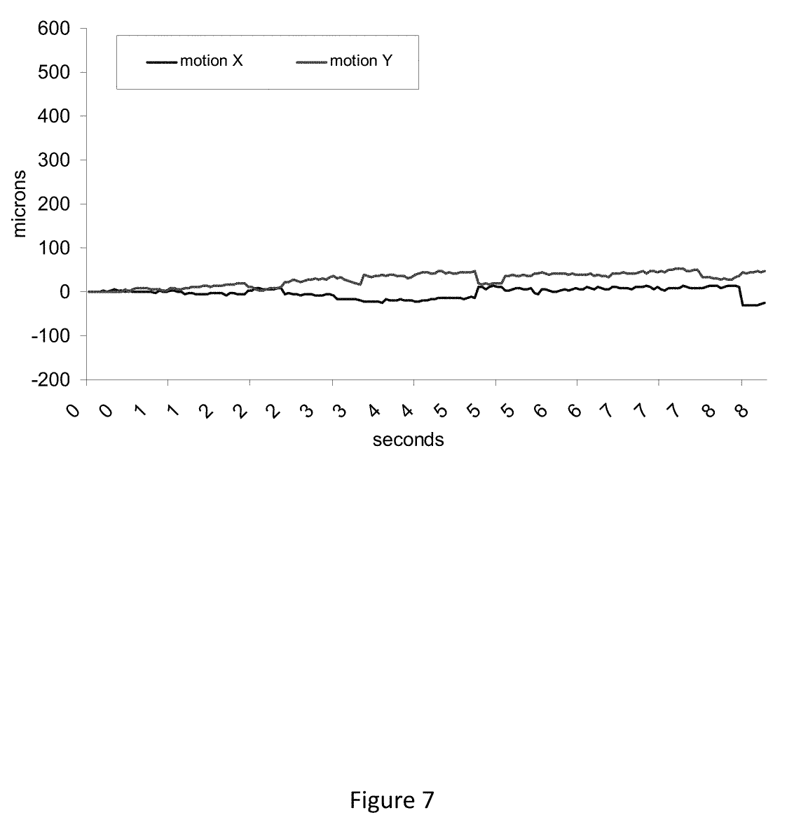

Accordingly, the ability to distinguish true eye motion from false eye motion increases the efficiency and accuracy of the system, which allows for a level of quality in real-time eye tracking unattainable with currently available systems. An example of the reduction in image motion when using the system is shown in FIG. 7. FIG. 7 is a graph showing tracked image motion in a diseased eye where an embodiment of the system employing WFSLO tracking is used for eye tracking. In this particular case, the image motion after the WFSLO tracking is about 1/15 (motion X) and 1/9 (motion Y) of the image motion without WFSLO tracking, as shown in FIG. 6.

Experiments with 20 subjects, 10 having normal eyes and 10 having diseased eyes, showed that tracking performance, in the form of residual image motion, in the direction of fast scan (i.e., motion X in the example) is significantly better than that from the direction of slow scan (i.e., motion Y). Therefore, in optical implementation, WFSLO fast/slow scanning should be perpendicular (rotated 90°) to AOSLO fast/slow scanning, i.e., the WFSLO fast axis should be perpendicular to the AOSLO fast axis, and the WFSLO slow axis should be perpendicular to the AOSLO slow axis. For example, if the WFSLO has fast/slow scanning in X/Y directions, then the AOSLO has fast/slow scanning in Y/X directions. If the WFSLO has fast/slow scanning in Y/X directions, then the AOSLO has fast/slow scanning in X/Y directions.

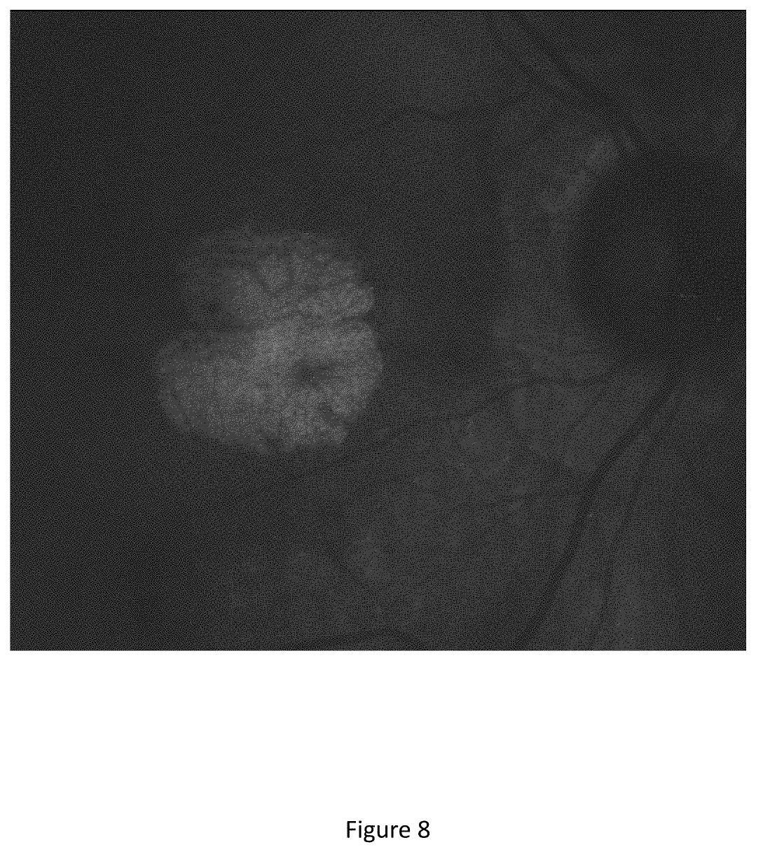

FIG. 8 is an example of the wide FOV, relatively low resolution, coarse-scale images that are used in the system and method of eye tracking. FIG. 8 is a single frame of an image from a WFSLO from the eye of a patient with cone-rod dystrophy. Individual live retinal images from a WFSLO typically contain a high percentage of low-contrast and dark regions, even if the optical system has been optimized. In a real-time image-based eye tracking system, where the tracking algorithm retrieves motion signals from real-time images to control one or more tracking mirrors, these low-contrast images can introduce artifacts or noise into the tracking signals. In the WFSLO image in FIG. 8, the resonant (fast) scanner scanned in the horizontal direction and the linear (slow) scanner scanned in the vertical direction. All notations between width and height are switched when the scanning direction is switched horizontally and vertically.

To obtain high-fidelity eye motion, the system and method tracks only blood vessels, and avoids the optic nerve disc because the optic disc is too rich in features. In general, a cross-correlation based tracking algorithm will fail when the optic nerve disc appears only on the reference image or only on the target image, but not when it appears in both images. Accordingly, the efficiency of the system and method is improved by not tracking the optic nerve disc.

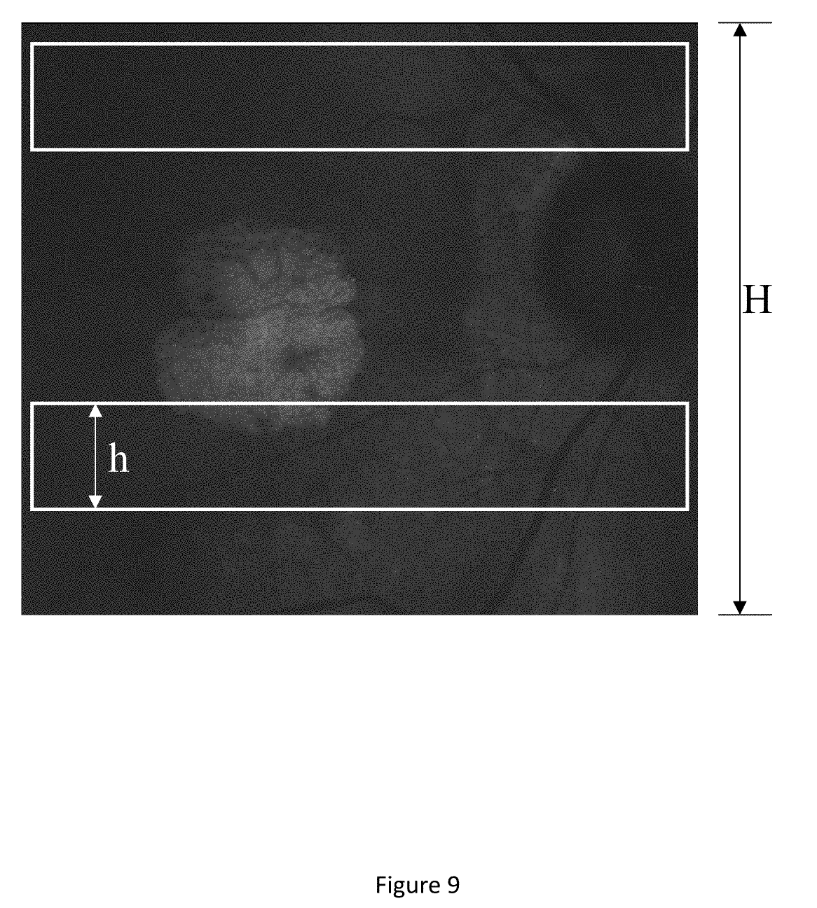

To achieve faster and smoother control for the tracking mirror, the field of view in the direction of slow scanning will be reduced to the height of the rectangle at faster frame rate, and the width of the image stays the same. For example, referring to FIG. 9, if the full image with height H has a frame rate f, and a smaller subset image with height h has frame rate F, these four parameters will satisfy the approximate equation,

F×H=f×h. (3)

The smaller image with height h that is captured at a high frame rate can be cropped from anywhere of the central part of the large, slow frame rate image, as long as the boundary of h does not run outside of H and the small image does not contain the optic nerve disc. The height h can be as small as possible, as long as the light power is under the ANSI safety level, and the small image contains enough features of blood vessels for cross-correlation. The height h can be set to no larger than ½ of H so that the h less frequently runs out of the boundary of H with fixational eye motions.

In one embodiment of an image-based tracking system, the large image with height H is used as a reference image and the small image with height h is used as a target image. A 2-D smoothing filter (e.g., Gaussian), followed by a 2D edge-detecting filter (e.g., Sobel) can be applied, if necessary, on both the reference image and the target image to retrieve the features of the blood vessels. A threshold can be applied on the filtered images to remove the artifacts caused by filtering, random noises, and/or a low-contrast background.

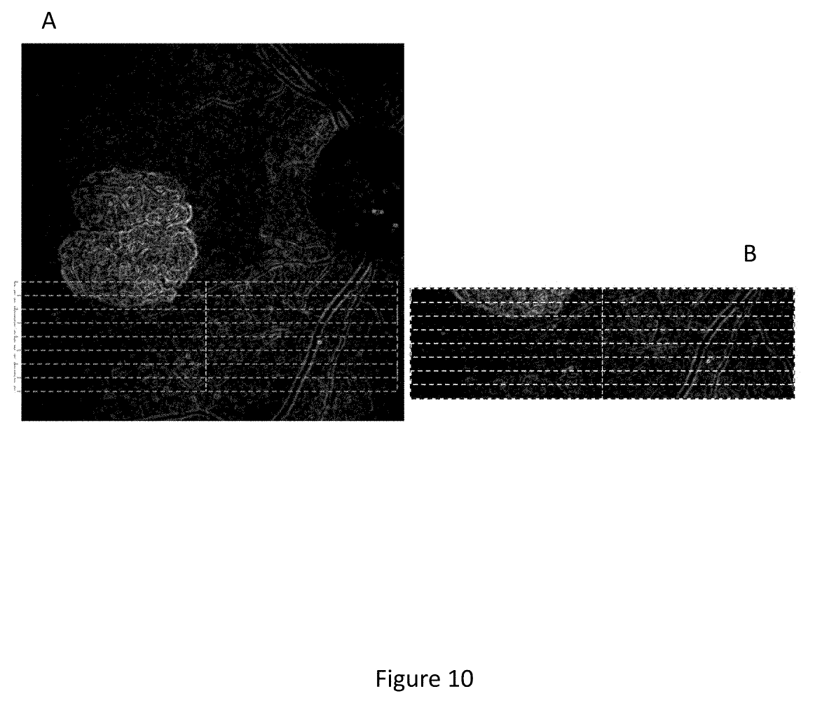

The method of image registration and eye-tracking involves cross-correlation between the reference and target images. As shown in FIG. 10, the reference image (8A) and the filtered target image (8B) are further divided into multiple strips with approximately the same width as the target image in FIG. 9, but with heights smaller than the height h in FIG. 9. Each strip in FIGS. 10A and 10B are further divided into two equally-sized sub-strips, i.e., one at the left and the other at the right, to aid in detecting eye torsion, which occurs frequently due to rotation of the eye or the head position. Cross-correlation can then be applied by comparing two corresponding strips, one from the reference image and one from the target image.

In an integrated eye tracking system, where the tracking mirror controlled by the SLO images can be used to dynamically steer the beam on another imaging system, such as an AOSLO or an OCT, relatively smooth motion from the tracking mirror is highly important. In one embodiment, smooth motion and control of the tracking mirror can be achieved as follows. The wide FOV SLO images are line-interleaved to achieve a doubled frame rate. With a doubled frame rate, the number of strips created per second in FIG. 10 is also doubled, and the update rate of the tracking mirror is doubled as well. A sub-pixel cross-correlation algorithm can be implemented to calculate eye motions from the SLO images. The optical resolution of a single pixel from the SLO system is usually on the order of tens of microns. A whole pixel of SLO motion applied on the tracking mirror will cause severe jitters on the AOSLO images, similar to microsaccades from human eyes. In one embodiment of the system and method, a digital low-pass filter can be applied on the motion traces to reduce unexpected spikes on the motion signals. In one embodiment, a high-resolution digital-to-analog converter (DAC) can be implemented to convert the (low-pass filtered) motion trace which is applied on the tracking mirror. In one embodiment, an analog low-pass filter can then be implemented after digital-to-analog conversion instead of, or in addition to, the digital low-pass filter.

Methods for rapidly re-locking the tracking of a subject's eye position after a blink or some other type of interference with eye image tracking are also described herein. Typically there are three statuses of fixational eye motion that must be considered during eye tracking: drift, blink, and microsaccade. Blinks can be discriminated by mean and standard deviation from individual image strips. When both mean and standard deviation of a strip drops below user-defined thresholds, this strip is treated as a blink frame, and the tracking mirror is suspended at its existing position. A microsaccade causes a single image strip to move several pixels in comparison to the previous strip. When multiple continuous strips move several pixels, the motion of the most recent strip is updated immediately on the tracking mirror. The number of multiple continuous strips required to cause an update on the tracking mirror can be determined by the user to balance tracking robustness and tracking accuracy. The update on tracking mirror is caused by a pulse signal to the tracking mirror to quickly adjust its status to compensate for a microsaccade. However, when only a single strip moves several pixels, it is not treated as a microsaccade strip, because this single motion is likely due to a miscalculation of the tracking algorithm as a result of minor variances or errors during cross-correlation between the target image strip and the reference image. In such a case, the position of the tracking mirror will be suspended at its current status. In motion associated with eye drift, the approach of using double frame rates and low-pass filters described above can be applied on the tracking mirror to control the tracking mirror smoothly.

In a multi-scale tracking system, e.g., the system shown in FIG. 3, WFSLO tracking and AOSLO tracking are implemented in conjunction with each other as follows. The WFSLO continues eye tracking as long as the location of the fixation target does not change. When a region of interest (ROI) is determined on the WFSLO images, AOSLO FOV is quickly, e.g., within 2-3 seconds, steered to this area and zoomed in to get high-resolution live videos from the retina. Eye tracking, or optical stabilization, is started by using the AOSLO imaging in combination with AOSLO digital registration. When there is a rotation of the eye or the head, the reference frame of the WFSLO has to be adjusted. However, currently available systems have no hardware to optically rotate imaging FOVs of AOSLO and WFSLO, and the amount of rotation is beyond their capability for the detection of rotation and translation. If eye motion of the target frame m relative to the original reference frame is

(xₘ,yₘ,θₘ) (4)

and due to difficult eye/head rotation, this target frame m has to be updated as a new reference frame, then the future frame n will cross correlate with this frame m, with motion

(dxₙ,dyₙ,dθₙ) (5)

The net eye motion of frame n relative to the original reference is then

(xₘ+dxₙ,yₘ+dyₙ,θₘ+dθₙ) (6)

This approach enables the WFSLO to continuously track eye location, so that AOSLO imaging becomes efficient in steering its FOV to any ROI as along as it is in the steering range. At a particular fixation target, all reference frames are saved in an imaging session and their positions are determined by Equations (4)-(6). If the imaging session is stopped temporarily, i.e., the subject takes a break during the procedure, the AOSLO tracking system picks out the most optimal frame from the existing reference frames for the next imaging session. The location of AOSLO imaging FOV is passed to the WFSLO and recorded on a WFSLO image. Each AOSLO video has a unique WFSLO image to record its imaging position and size of FOV. The WFSLO notifies its tracking status to the AOSLO, e.g., microsaccade, blink, or tracking failure. In addition, the AOSLO notifies its status to the WFSLO, e.g., data recording and AOSLO tracking. Further, the WFSLO eye-tracking updates a new reference frame when the fixation target changes to a new location.

The system can use a number of different approaches to achieve smooth and robust control for the one or more tracking mirrors (i.e., mirrors M2 and M3 in FIG. 3). In the systems in FIGS. 3 through 5, a tracking algorithm is used to implement the control of M2 in the control loop of M1-WFSLO-M2. The control signals for M2 come from the real-time images of the WFSLO with cross-correlation technology. In the system of FIG. 3, a second control loop, i.e., the closed control loop between AOSLO and M3 is also used in the image-based tracking method.

Referring again to FIG. 3, light from the retina will be split into two channels via beam splitter M1, wherein one channel is sent to the wide FOV system (WFSLO), and the other channel to a tracking mirror M2. The light is then further directed to a second tracking mirror M3, and then related to the small FOV system (AOSLO). To reduce latency and increase accuracy on controlling the tracking mirror, the tracking mirror must be updated fast enough, e.g., every millisecond, to track eye motion. Accordingly, the system can also require a suitable electronics system for image processing.

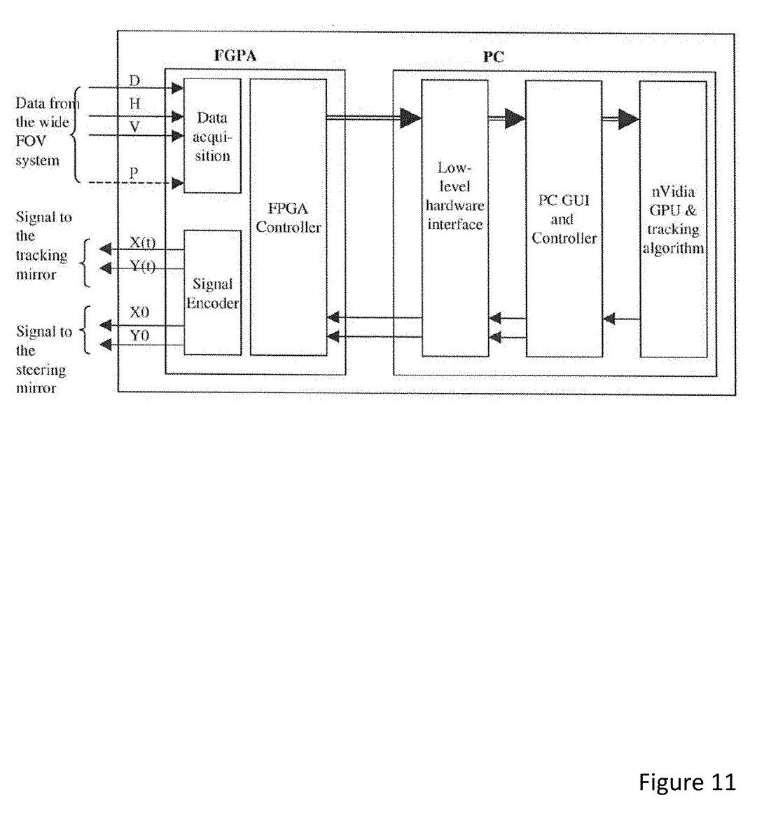

A schematic diagram of an exemplary embodiment of the electronics system for the wide FOV system is shown in FIG. 11. In this system architecture, there are two modules: a FPGA module and a PC module. The FPGA module is responsible for real-time data acquisition from the optical system, flexible data buffering between FPGA and the host PC via a programmable controller, such as a PCle controller, and data encoding to one or multiple D/A converters to control external devices such as the one or more tracking mirrors and any steering mirrors. Images from the wide FOV system can be in 1) analog format with analog data, H-sync, and V-sync, or 2) digital format with digital data, H-sync, V-sync, and pixel clocks. In analog format, an A/D converter is needed to digitize the images so that they can be sent to the FPGA. In digital format, FPGA can be programmed to sample parallel or serial digital data from the wide FOV optical system. In both cases, the digitized H-sync, V-sync and pixel clock can be used as common clocks throughout the entire FPGA application for buffering data from FPGA to PC through PCle interface. These three clocks are also used to synchronize D/A converters that output eye motion signals to the one or more tracking mirrors and control any steering mirrors. The FPGA are programmed to control any resolution of off-shelf A/D and D/A converters, from 8 bits to 16 bits or higher resolution.

The PC module is responsible for collecting images from the FPGA, sending the images to a graphics processing unit (GPU) for data processing, and then uploading eye motion signals and other control signals to the FPGA. The PC GUI and controller manage the hardware interface between the PC and the FPGA, the GPU image registration algorithm, and the data flow between the FPGA, the PC CPU, and the GPU. In various embodiments, the GPU is a GPU manufactured by nVidia, or any other suitable GPU as would be understood by a person skilled in the art. In one embodiment, the FPGA is a Xilinx FPGA board (ML506, ML605, or newer modules, Xilinx, San Jose). The selection of ML506 or ML 605 can depend on the format of images from the optical system, i.e., the ML506 can be used for analog data and the ML605 can be used for digital data. However, the FGPA can be any suitable board known in the art.

The architecture of the small FOV system can be similar to that of the wide FOV system described above, except that only one steering mirror or set of steering mirrors is controlled, and the signals can come from either the WFSLO software or the AOSLO software. However, in order to have maximum flexibility for additional functionality, the same Xilinx FPGA board (ML506 or ML605) used in the wide FOV system can be used in the small FOV system. This additional functionality can include, but is not limited to: real-time stabilized beam control to the retina, allowing for laser surgery with operation accuracy in hundreds of nanometers on the living retina; delivery of highly controllable image patterns to the retina for scientific applications; and the real-time efficient montaging of retinal images.

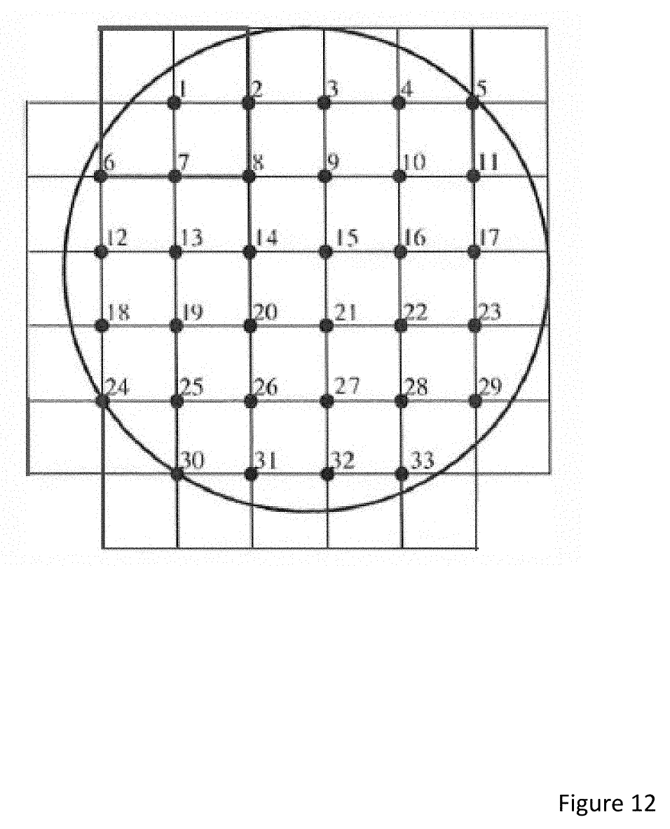

For example, FIG. 12 is a drawing representing the process of real-time retinal montaging. The circled area is the retina covered by the wide FOV system with low spatial resolution, and an area equivalent to four squares is covered by the small FOV system with high spatial resolution. To achieve a high-resolution image montage from the retina, the two systems can be programmed to direct the steering mirror to the locations of the dots with labels 1, 2, 3, etc., one at a time, wherein the four squares surrounding the targeted dot is covered by the small FOV system. In each location, the tracking mirror compensates for large and small eye motion, and the registration algorithm on the small FOV system removes the residual image motions in real time, and then registers the images. In one embodiment, the software and hardware needs only about 5-10 seconds to register images in each location to achieve a high signal-to-noise ratio (SNR) averaged image. The steering mirror can automatically be directed to the next location after the current one is finished. When the steering mirror sweeps through all predetermined locations (i.e., 33 in the example shown in FIG. 12), the software will automatically generate a large montage of the retina image. In such an embodiment, imaging of adjacent locations must be overlapped. The amount of overlapping required to maintain eye tracking depends on the residual eye motion on the small FOV system.

In one aspect, the system and method is an improvement over currently available technologies in that it can be used to process 512×512 pixel (or equivalent sized) warped images at 120 frames per second with high accuracy on a moderate GPU, for example an nVidia GTX560. The method takes advantage of the parallel processing features of GPUs, unlike currently available systems and methods that process less than 30 frames/second using a same or similar GPU.

The system and method can be used to perform the following: real-time image registration from a small and wide FOV SLO running at 30 frames/second or higher, e.g., in one embodiment, the frame rate can be 60 frames/second; real-time control of a tracking mirror to remove large eye motion on the small FOV SLO (1-2 degrees), by applying real-time eye motion signals from a large FOV SLO (10-30 degrees) every millisecond; and compensation of eye motion from an OCT in high accuracy with millisecond latency by applying real-time eye motion signals from a large FOV SLO (10-30 degrees) on the scanners of the OCT.

The method of image registration generally includes the following steps: 1) choose a reference frame, and divide it into several strips to account for image distortion; 2) retrieve a target frame, and also divide the target frame into the same number of strips as the reference frame; 3) perform cross-correlation between the reference strip and the target strip to calculate the motion of each target strip; and 4) register the target frame to the reference frame accounting for all motions of the target strips.

The speed and accuracy of the cross-correlation step, i.e., step 3, will determine the overall speed and accuracy of the image registration. Previous approaches to this step described in the prior art are not fast enough to enable image registration in real time. One reason for the lack of speed in these approaches is that they do not start the image registration algorithm until a whole frame is received by the host PC. This frame-level registration results in significant latency in controlling external devices such as scanners and/or tracking mirrors. For example, the shortest latency in such an approach is the frame rate of an imaging system, which can be about 33 milliseconds on a 30 frames/second system. Accordingly, when the computational latency from the GPU, CPU, and other processors are included, the total latency is generally significantly greater than 33 milliseconds.

The method can be used to perform fast, real-time image registration by dramatically improving processing speed over currently known approaches. The method is based on an algorithm that starts image registration as soon as a new strip from a target image is received by the host PC, instead of waiting for a whole frame to be delivered, as in current approaches. For example, a 520×544 image can be divided into 34 strips, each with a size of 520×16 pixels. Each strip is sent from the device to the host PC, which immediately sends it to the GPU where the motion of the strip is calculated.

On a testing benchmark with a nVidia GTX560 GPU, the computational time for processing each strip is about 0.17 millisecond. The dominant latency is from sampling the 520×16 strip which takes about 1.0 millisecond on a 30 frames/second system. Therefore, the total latency from input data to sending an output motion signal is about 1.5 milliseconds. In one embodiment, the sampling latency can be further reduced if the frame rate of an imaging system is increased.

In another aspect of the method, the algorithm implemented in the GPU to achieve a computational time of 0.17 milliseconds per strip is also a significant improvement over the known art. Currently available methods mix parallel and serial processing on the GPU, resulting in busy data buffering between GPU and the host PC. To fully take advantage of the GPU computational capacity, the method uses the GPU for parallel processing only, and converts all serial processing into parallel processing on the GPU. Further, the data communication between the GPU and the host PC is minimized. Specifically, to achieve optimal speed, raw image data is sent only once to the GPU. The GPU then performs all required processing in parallel, and returns only three parameters from the GPU to the host PC: the correlation coefficient and translations x and y. Further still, speed is improved by use the GPU shared memory and/or texture as much as possible, while avoiding the GPU global memory.

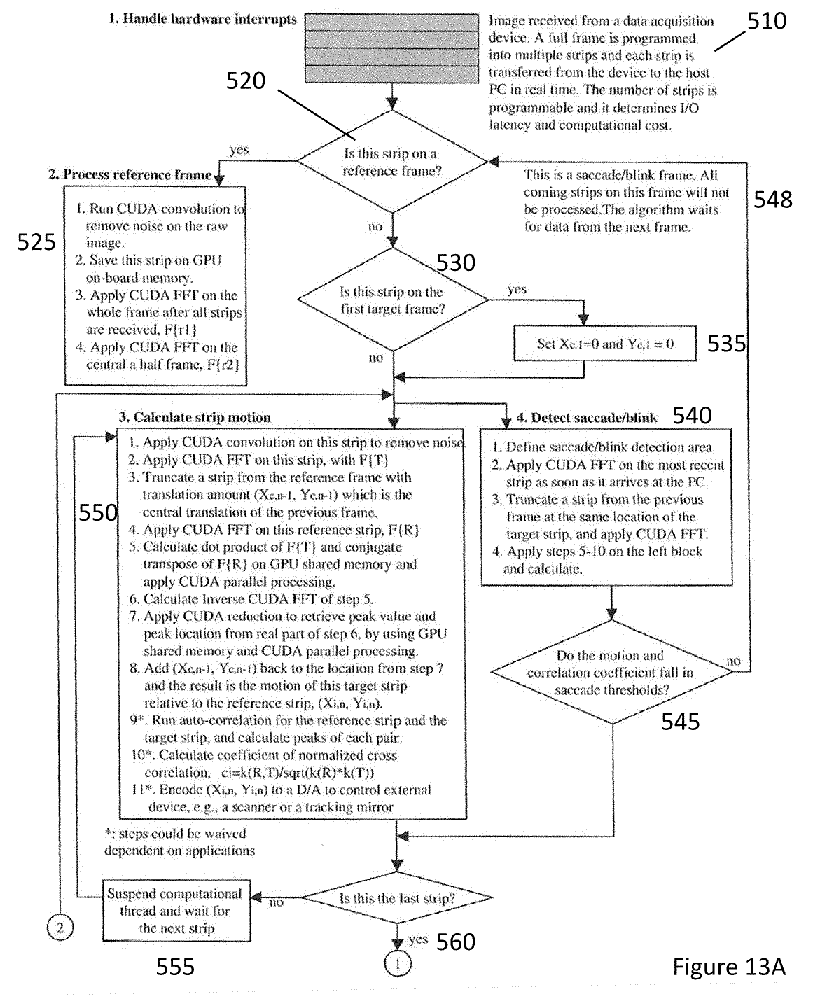

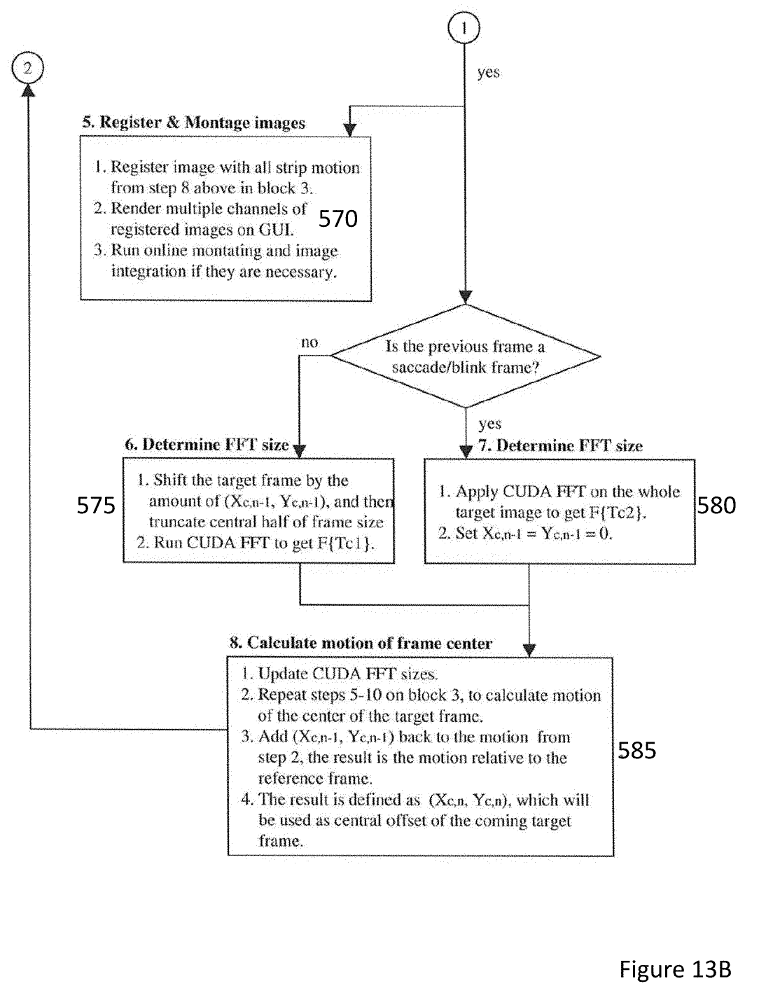

A flow chart of the algorithm for one embodiment of the method is shown in FIGS. 13A and 13B. First, an image is acquired from a data acquisition device, e.g., an AOSLO or wide FOV SLO (step 510). The image, i.e., a single frame, is divided into multiple strips, and each strip is transferred from the device to the host PC in real time. In a preferred embodiment, as previously described herein, each strip is sent to the host PC immediately upon being generated instead of waiting for the entire frame to be generated and then divided into strips. The number of strips that the image is divided into is a programmable variable. The number of strips chosen can affect the I/O latency and computational cost.

If a strip is designated as coming from a reference frame (520) the strip will be processed using a reference frame protocol (525). Specifically, step 525 includes running a compute unified device architecture (CUDA) model implemented on the GPU, wherein noise is removed on the raw image, the strip saved on the GPU, and a CUDA fast Fourier transform (FFT) is applied to the whole frame or half frame. If a strip is not designated as coming from a reference frame, the strip is queried whether it is a strip on the first target frame (530). If the strip is on the first target frame, X_{c,1} and Y_{c,1} are each set to zero (535). If the strip is not on the first target frame, two protocols are run on the strip simultaneously. Specifically, a saccade/blink detection protocol is run (540) in conjunction with a protocol for calculating the strip motion (550). If a saccade or blink is detected (545), processing of all strips coming from this frame will be stopped and the algorithm will wait for the next frame (548). If a saccade or blink is not detected, the strip motion processing continues for the entire frame (550 & 555) until the last strip is received (560). After the last strip of a frame is received, the image is registered and, if necessary, montaged (570). Further, the FFT size is determined accordingly, based on whether the previous frame is a saccade/blink frame (580) or not a saccade/blink frame (575). The motion of the frame center is then calculated, which can be used to offset the next target frame as needed (585).

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A scanning laser ophthalmoscopy system, comprising:
a wide field of view scanning laser ophthalmoscope (SLO) having a controller,
a beam splitter,
a first tracking mirror,
a second tracking mirror, and
a small field of view imaging apparatus having a controller,
wherein the beam splitter is configured to split a beam of light backscattered from a subject's eye into a first beam and a second beam, such that the first beam is incident on the wide field of view SLO, and the second beam is incident on the first tracking mirror; the second beam is further reflected by the first tracking mirror onto the small field of view apparatus via the second tracking mirror; the wide field of view SLO controller is communicatively coupled with the first tracking mirror; the small field of view apparatus controller is communicatively coupled with the second tracking mirror; and the system compensates for a motion of the subject's eye during eye tracking by moving the first tracking mirror via the wide field of view SLO controller, moving the second tracking mirror via the small field of view apparatus controller, or both.

2. The system of claim 1, wherein the small field of view apparatus is a small field of view SLO.

3. The system of claim 1, wherein the small field of view apparatus is an adaptive optics scanning light ophthalmoscope (AOSLO).

4. The system of claim 1, wherein the small field of view apparatus is an optical coherence tomography (OCT) apparatus.

5. The system of claim 1, wherein the field of view of the wide field of view SLO is in the range of about 10 to 30 degrees.

6. The system of claim 1, wherein the field of view of the small field of view apparatus is in the range of about 1 to 2 degrees.

7. The system of claim 1, wherein moving the tracking mirror via the wide field of view SLO controller can compensate for an eye motion of about ±3°.

8. The system of claim 1, wherein moving the second tracking mirror via the small field of view apparatus controller can compensate for an eye motion of about ±3°.

9. (canceled)

10. A scanning laser ophthalmoscopy system, comprising:
a wide field of view scanning laser ophthalmoscope (SLO),
a beam splitter,
a small field of view imaging apparatus,
a controller communicatively coupled with the wide field of view SLO and the small field of view imaging apparatus, and
a tracking mirror communicatively coupled with the controller,
wherein the beam splitter is configured to receive a beam of light backscattered from a subject's eye; the beam splitter splits the beam of light into a first beam and a second beam, such that the first beam is incident on the wide field of view SLO, and the second beam is incident on the tracking mirror; and the system compensates for a motion of the subject's eye during eye tracking by moving the tracking mirror via the controller.

11. The system of any of claim 10, wherein the small field of view apparatus is a small field of view SLO.

12. The system of any of claim 10, wherein the small field of view imaging apparatus is an adaptive optics scanning light ophthalmoscope (AOSLO).

13. The system of any of claim 10, wherein the small field of view apparatus is an optical coherence tomography (OCT) apparatus.

14. The system of any of claim 10, wherein the field of view of the wide field of view SLO is in the range of about 10 to 30 degrees.

15. The system of any of claim 10, wherein the field of view of the small field of view imaging apparatus is in the range of about 1 to 2 degrees.

16. The system of any of claim 10, wherein moving the tracking mirror via the controller can compensate for an eye motion of about ±6°.

17. A method of real-time eye tracking using a small field of view imaging system, comprising:
obtaining a reference image of at least a portion of a subject's retina,
dividing at least a portion of the reference image into one or more strips,
sending the one or more reference strips to a microprocessor,
obtaining a high resolution target image of at least a portion of the subject's retina,
dividing at least a portion of the target image into one or more strips,
sending the one or more target strips to a host microprocessor,
sending the one or more target strips from the host microprocessor to a graphics microprocessor, wherein each target strip is correlated with a reference strip,
returning at least one output parameter from the graphics microprocessor to the host microprocessor, wherein the at least one output parameter corresponds to the motion of the target strip compared to the reference strip, and
registering the target image to the reference image based on the at least one output parameter.

18. The method of claim 17, wherein the at least one output parameter is a correlation coefficient.

19. The method of claim 17, wherein the at least one output parameter is an x translation or a y translation.

20. The method of claim 17, wherein the time for correlating each target strip with a reference strip is less than about 0.2 milliseconds.

21. The method of claim 17, wherein the total latency time from obtaining the reference image to registering the target frame to the reference image is less than about 2 milliseconds.

22. The method of claim 17, wherein the reference image is obtained from a wide field of view SLO.

23. The method of claim 17, wherein the target image is obtained from a small field of view imaging apparatus.

24. The method of claim 23, wherein the small field of view imaging apparatus is a small field of view SLO.

25. The method of claim 23, wherein the small field of view imaging apparatus is an AOSLO.

26. The method of claim 23, wherein the small field of view imaging apparatus is an OCT apparatus.

27. The method of claim 17, wherein the target image is not registered to the reference image if the target image corresponds to a saccade or blink of the subject's eye.

28. The method of claim 17, wherein the reference image is obtained from a wide field of view SLO, wherein the target image is obtained from a small field of view imaging apparatus, and wherein the direction of the wide field of view SLO fast-scanning axis is perpendicular to the small field of view apparatus fast-scanning axis, and the wide field of view SLO slow-scanning axis is perpendicular to the small field of view apparatus slow-scanning axis.
